(19) United States
(12) Patent Application Publication
(10) Pub. No.: US 2016/0192686 A1
(43) Pub. Date: Jul. 7, 2016

(54) COHESIVE LIQUID BOLUS COMPRISING MOLECULES PROVIDING VISCO-ELASTICITY

(71) Applicant: NESTEC S.A., Vevey (CH)

(72) Inventors: Adam Burbidge, Arzier (CH); Jan Engmann, Lausanne (CH); Simina Popa Nita, Morges (CH); Elizabeth Offord Cavin, Montreux (CH)

(21) Appl. No.: 14/897,834

(22) PCT Filed: Jun. 4, 2014
(86) PCT No.: PCT/EP2014/061590

§ 371 Date: Dec. 11, 2015

(30) Foreign Application Priority Data
Jun. 14, 2013 (EP) 13172158.1

(51) Int. Cl. A23L 1/05; A61K 9/00; A61K 31/737; A61K 31/728; A61K 38/39; A23L 1/056; A23L 1/0562

(52) U.S. Cl. CPC A23L 1/296; A23L 1/3002; A23L 1/05; A61K 31/728; A61K 9/0053; A61K 31/737; A61K 38/39

(57) ABSTRACT

This invention relates to a cohesive thin liquid nutritional product comprising an aqueous solution of at least one food grade biopolymer selected from a group of molecules providing visco-elasticity, to the use of said product for promoting safer swallowing of food boluses in dysphagic patients and to a method for preparing the product.

### FIELD OF THE INVENTION

This invention relates to a cohesive thin liquid product comprising an aqueous solution of at least one food grade biopolymer and to the use of said product for promoting safer swallowing of food boluses in patients having difficulty in swallowing. The invention further relates to a method for preparing such a cohesive thin liquid product.

### BACKGROUND OF THE INVENTION

Dysphagia is the medical term for the symptom of difficulty in swallowing. Epidemiological studies estimate a prevalence rate of 16% to 22% among individuals over 50 years of age.

Esophageal dysphagia affects a large number of individuals of all ages, but is generally treatable with medications and is considered a less serious form of dysphagia. Esophageal dysphagia is often a consequence of mucosal, mediastinal, or neuromuscular diseases. Mucosal (intrinsic) diseases narrow the lumen through inflammation, fibrosis, or neoplasia associated with various conditions (e.g., peptic stricture secondary to gastroesophageal reflux disease, esophageal rings and webs [e.g., sideropenic dysphagia or Plummer-Vinson syndrome], esophageal tumors, chemical injury [e.g., caustic ingestion, pill esophagitis, sclerotherapy for varices], radiation injury, infectious esophagitis, and eosinophilic esophagitis). Mediastinal (extrinsic) diseases obstruct the esophagus by direct invasion or through lymph node enlargement associated with various conditions (tumors [e.g., lung cancer, lymphoma], infections [e.g., tuberculosis, histoplasmosis], and cardiovascular [dilated auricula and vascular compression]). Neuromuscular diseases may affect the esophageal smooth muscle and its innervation, disrupting peristalsis or lower esophageal sphincter relaxation, or both, commonly associated with various conditions (achalasia [both idiopathic and associated with Chagas disease], scleroderma, other motility disorders, and a consequence of surgery [i.e., after fundoplication and antireflux interventions]). It is also common for individuals with intraluminal foreign bodies to experience acute esophageal dysphagia.

Oral pharyngeal dysphagia, on the other hand, is a very serious condition and is generally not treatable with medication. Oral pharyngeal dysphagia also affects individuals of all ages, but is more prevalent in older individuals. Worldwide, oral pharyngeal dysphagia affects approximately 22 million people over the age of 50. Oral pharyngeal dysphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex. It is also common for individuals with progressive neuromuscular diseases, such as Parkinson's Disease, to experience increasing difficulty in swallowing initiation. Representative causes of oropharyngeal dysphagia include those associated neurological illnesses (brainstem tumors, head trauma, stroke, cerebral palsy, Guillain-Barre syndrome, Huntington's disease, multiple sclerosis, polio, post-polio syndrome, Tardive dyskinesia, metabolic encephalopathies, amyotrophic lateral sclerosis, Parkinson's disease, dementia), infectious illnesses (diphtheria, botulism, Lyme disease, syphilis, mucositis [herpetic, cytomegalovirus, candida, etc.]), autoimmune illnesses (lupus, scleroderma, Sjogren's syndrome), metabolic illnesses (amyloidosis, cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects [e.g., chemotherapy, neuroleptics, etc.], post surgical muscular or neurogenic, radiation therapy, corrosive [pill injury, intentional]), and structural illnesses (cricopharyngeal bar, Zenker's diverticulum, cervical webs, oropharyngeal tumors, osteophytes and skeletal abnormalities, congenital [cleft palate, diverticulae, pouches, etc.]).

Dysphagia is not generally diagnosed although the disease has major consequences on patient health and healthcare costs. Individuals with more severe dysphagia generally experience a sensation of impaired passage of food from the mouth to the stomach, occurring immediately after swallowing. Among community dwelling individuals, perceived symptoms may bring patients to see a doctor. Among institutionalized individuals, health care practitioners may observe symptoms or hear comments from the patient or his/her family member suggestive of swallowing impairment and recommend the patient be evaluated by a specialist. As the general awareness of swallowing impairments is low among front-line practitioners, dysphagia often goes undiagnosed and untreated. Yet, through referral to a swallowing specialist (e.g., speech language pathologist), a patient can be clinically evaluated and dysphagia diagnosis can be determined.

Severity of dysphagia may vary from: (i) minimal (perceived) difficulty in safely swallowing foods and liquids, (ii) an inability to swallow without significant risk for aspiration or choking, and (iii) a complete inability to swallow. Commonly, the inability to properly swallow foods and liquids may be due to food boluses being broken up into smaller fragments, which may enter the airway or leave unwanted residues in the oropharyngeal and/or esophageal tract during the swallowing process (e.g., aspiration). If enough material enters the lungs, it is possible that the patient may drown on the food/liquid that has built up in the lungs. Even small volumes of aspirated food may lead to bronchopneumonia infection, and chronic aspiration may lead to bronchiectasis and may cause some cases of asthma.

“Silent aspiration,” a common condition among elderly, refers to the aspiration of oropharyngeal contents such as secretions, food, or liquid due to a lack of pharyngeal reflex in the absence of cough, throat clearance or distress. People may compensate for less-severe swallowing impairments by self-limiting the diet. The aging process itself, coupled with chronic diseases such as hypertension or osteoarthritis, predisposes elderly to (subclinical) dysphagia that may go undiagnosed and untreated until a clinical complication such as pneumonia, dehydration, malnutrition (and related complications) occurs.

Pneumonia is a common clinical consequence of dysphagia. The condition often requires acute hospitalization and emergency room visits. Among those that develop pneumonia due to aspiration, the differential diagnosis of ‘aspiration pneumonia’ is not necessarily indicated as a result of current care practices. Based on U.S. healthcare utilization surveys from recent years, pneumonia accounted for over one million hospital discharges and an additional 392,000 were attributable to aspiration pneumonia. Individuals who have general pneumonia as the principal diagnosis have a mean 6 day hospital length of stay and incur over $18,000 in costs for hospital care. It is expected that aspiration pneumonia would carry higher costs for hospital care, based on a mean 8 day length of hospital stay. Pneumonia is life threatening among persons with dysphagia, the odds of death within 3 months is about 50% (van der Steen et al. 2002). In addition, an acute insult such as pneumonia often initiates the downward spiral in health among elderly. An insult is associated with poor intakes and inactivity, resulting in malnutrition, functional decline, and frailty. Specific interventions (e.g., to promote oral health, help restore normal swallow, or reinforce a swallow-safe bolus) would benefit persons at risk for (due to aspiration of oropharyngeal contents, including silent aspiration) or experiencing recurrent pneumonia.

Similar to pneumonia, dehydration is a life-threatening clinical complication of dysphagia. Dehydration is a common co-morbidity among hospitalized individuals with neurodegenerative diseases (thus, likely to have a swallowing impairment). The conditions of Alzheimer's disease, Parkinson's disease, and multiple sclerosis account for nearly 400,000 U.S. hospital discharges annually, and up to 15% of these patients suffer dehydration. Dehydration as the principal diagnosis is associated with a mean 4 day length of hospital stay and over $11,000 in costs for hospital care. Nevertheless, dehydration is an avoidable clinical complication of dysphagia.

Malnutrition and related complications (e.g., [urinary tract] infections, pressure ulcers, increased severity of dysphagia [need for more-restricted food options, tube feeding, and/or PEG placement and reduced quality of life], dehydration, functional decline and related consequences [falls, dementia, frailty, loss of mobility, and loss of autonomy]) can arise when swallowing impairment leads to fear of choking on food and liquids, slowed rate of consumption, and self-limited food choices. If uncorrected, inadequate nutritional intake exacerbates dysphagia as the muscles that help facilitate normal swallow weaken as physiological reserves are depleted. Malnutrition is associated with having a more than 3-times greater risk of infection. Infections are common in individuals with neurodegenerative diseases (thus, likely to have a chronic swallowing impairment that jeopardizes dietary adequacy). The conditions of Alzheimer's disease, Parkinson's disease, and multiple sclerosis account for nearly 400,000 U.S. hospital discharges annually, and up to 32% of these patients suffer urinary tract infection.

Moreover, malnutrition has serious implications for patient recovery. Malnourished patients have longer length of hospital stay, are more likely to be re-hospitalized, and have higher costs for hospital care. Malnutrition as the principal diagnosis is associated with a mean 8 day length of hospital stay and nearly $22,000 in costs for hospital care. Furthermore, malnutrition leads to unintentional loss of weight and predominant loss of muscle and strength, ultimately impairing mobility and the ability to care for oneself. With the loss of functionality, caregiver burden becomes generally more severe, necessitating informal caregivers, then formal caregivers, and then institutionalization. However, malnutrition is an avoidable clinical complication of dysphagia.

Among persons with neurodegenerative conditions (e.g., Alzheimer's disease), unintentional weight loss as a marker of malnutrition precedes cognitive decline. In addition, physical activity can help stabilize cognitive health. Thus, it is important to ensure nutritional adequacy among persons with neurodegenerative conditions to help them have the strength and endurance to participate in regular therapeutic exercise and guard against unintentional weight loss, muscle wasting, loss of physical and cognitive functionality, frailty, dementia, and progressive increase in caregiver burden.

The economic costs of dysphagia are associated with hospitalization, re-hospitalization, loss of reimbursement due to pay for performance (“P4P”), infections, rehabilitation, loss of work time, clinic visits, use of pharmaceuticals, labor, care taker time, childcare costs, quality of life, increased need for skilled care. Dysphagia and aspiration impact quality of life, morbidity and mortality. Twelve-month mortality is high (45%) among individuals in institutional care who have dysphagia and aspiration. The economic burden of the clinical consequences arising from lack of diagnosis and early management of dysphagia are significant.

In sum, the consequences of untreated or poorly managed oral pharyngeal dysphagia can be severe, including dehydration, malnutrition leading to dysfunctional immune response, and reduced functionality, airway obstruction with solid foods (choking), and airway aspiration of liquids and semi-solid foods, promoting aspiration pneumonia and/or pneumonitis. Severe oral pharyngeal dysphagia may require nutrition to be supplied by tube feeding.

Mild to moderate oral pharyngeal dysphagia may require the texture of foods to be modified in order to minimize the likelihood of choking or aspiration. This may include the thickening of liquids and/or pureeing of solid foods.

A known treatment for beverages and liquid foods is to increase the viscosity of the food/beverage by adding starch or gum thickeners. Such thickening is thought to improve bolus control and timing of swallowing. It is, however, often disliked by patients because of the extra swallowing effort and may also leave residues at high levels of viscosity. For solid foods, pureed diets are often described when problems with mastication and swallowing of solid pieces occur in patients. However, these pureed diets may lack the natural cohesiveness that saliva provides to “real” food boluses.

Therefore, and considering the prevalence of dysphagia, possible complications related thereto, and the costs associated with same, there is still a need for providing an improved method for treating swallowing disorders, which method can minimize the risk of standard bolus therapy, promotes safer swallowing of food boluses and prevents or treats the clinical complications of dysphagia in patients suffering from aspiration. Such a method would improve the lives of a large and growing number of persons with swallowing impairments. Specific interventions (e.g., to promote oral health, help restore normal swallow, or reinforce a swallow-safe bolus) can enable persons to eat orally (vs. being tube fed and/or requiring PEG placement) and experience the psycho-social aspects of food associated with general well being while guarding against the potentially negative consequences that result from lack of adequate swallowing ability. Improvements in the intake of nutrition by dysphagic patients may also enable such patients to swallow a wider variety of food and beverage products safely and comfortably, which may lead to an overall healthier condition of the patient and prevent further health-related decline.

### SUMMARY OF THE INVENTION

Therefore, the present disclosure provides improved nutritional products for promoting safer swallowing of food boluses in patients with swallowing disorders including, for example, dysphagia. These products effectively prevent bolus penetration and aspiration through modification of rheological properties of foods and beverages.

Accordingly, in a first aspect, the invention relates to a nutritional product, comprising an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from molecules providing visco-elasticity.

In a preferred embodiment of the first aspect of the invention, the visco-elasticity providing molecules are selected from the group consisting of hyaluronic acid, glucosamine sulphate, chondroitin sulphate, collagen, collagen peptides and combinations thereof.

In a further preferred embodiment of the first aspect of the invention, the shear viscosity is at least about 1 mPas, preferably from 5 to 45 mPas, more preferably from 10 to 40 mPas, and most preferably from 20 to 30 mPas, when measured at a shear rate of 50 s⁻¹.

In another preferred embodiment of the first aspect of the invention, the relaxation time is less than about 2000 ms, preferably from about 20 ms to about 1000 ms, more preferably from about 50 ms to about 500 ms, and most preferably from about 100 ms to about 200 ms, at a temperature of 20° C.

It is further preferred that in the first aspect of the invention the filament diameter of the nutritional product decreases less than linearly, and preferably exponentially in time during a CaBER experiment.

In a further preferred embodiment of the first aspect of the invention, the aqueous solution comprises the at least one food grade biopolymer in a concentration of from at least 0.01 wt % to 25 wt %, preferably from at least 0.1 wt % to 15 wt %, and most preferably from at least 1 wt % to 10 wt %.

A further preferred embodiment relates to the nutritional product of the first aspect of the invention in diluted form, preferably in an aqueous dilution ranging from 2:1 to 50:1, more preferably from 3:1 to 20:1 and most preferably from 5:1 to 10:1.

In a yet further preferred embodiment of the first aspect of the invention, the nutritional product comprises at least one further food grade biopolymer selected from the group consisting of botanical hydrocolloids, microbial hydrocolloids, animal hydrocolloids, algae hydrocolloids and any combination thereof. It is preferred that the algae hydrocolloids are selected from the group consisting of agar, carrageenan, alginate, or any combinations thereof. In another preferred embodiment, the microbial hydrocolloids are selected from the group consisting of xanthan gum, gellan gum, curdlan gum, or any combinations thereof. In a further preferred embodiment, the botanical hydrocolloids are selected from plant-extracted gums, plant-derived mucilages, or combinations thereof.

In a particularly preferred embodiment of the first aspect of the invention, the nutritional product comprises at least one further food grade biopolymer selected from plant-extracted gums, plant-derived mucilages, or combinations thereof. Preferably, the plant-extracted gums are selected from the group consisting of okra gum, konjac mannan, tara gum, locust bean gum, guar gum, fenugreek gum, tamarind gum, cassia gum, acacia gum, gum ghatti, pectins, modified celluloses (e.g., carboxymethyl cellulose, methyl cellulose, hydroxylpropyl methyl cellulose, hydroxypropyl cellulose), tragacanth gum, karaya gum, or any combinations thereof. It is mostly preferred that the plant-extracted gum is okra gum. In another preferred embodiment, the plant-derived mucilages are selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, or combinations thereof. It is mostly preferred that the plant-derived mucilage is kiwi fruit mucilage and/or cactus mucilage.

In another particularly preferred embodiment of the first aspect of the invention, the nutritional product comprises at least one further food grade biopolymer selected from okra gum and/or kiwi fruit mucilage, or a combination thereof.

A yet further preferred embodiment of the invention relates to the nutritional product of the above first aspect in administrable form selected from the group consisting of a nutritional formulation, a pharmaceutical formulation, a nutritional supplement, a dietary supplement, a functional food, a beverage product, a full meal, a nutritionally complete formula, and combinations thereof.

Another preferred embodiment of the invention relates to the nutritional product of the above first aspect for use in treating a swallowing disorder in a patient in need of same.

A further preferred embodiment of the invention relates to the nutritional product of the above first aspect for use in promoting safe swallowing of nutritional products in a patient in need of same.

A yet further preferred embodiment of the invention relates to the nutritional product of the above first aspect for use in mitigating the risks of aspiration during swallowing of nutritional products in a patient in need of same.

In a second aspect, the invention relates to a method for making a nutritional product, the method comprising providing an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from a group of molecules providing visco-elasticity.

In a preferred embodiment of the second aspect of the invention, the group of visco-elasticity providing molecules comprises hyaluronic acid, glucosamine sulphate, chondroitin sulphate, collagen, collagen peptides and combinations thereof.

In a further preferred embodiment of the second aspect of the invention, the shear viscosity is at least about 1 mPas, preferably from 5 to 45 mPas, more preferably from 10 to 40 mPas, and most preferably from 20 to 30 mPas, when measured at a shear rate of 50 s⁻¹.

In another preferred embodiment of the second aspect of the invention, the relaxation time is less than about 2000 ms, preferably from about 20 ms to about 1000 ms, more preferably from about 50 ms to about 500 ms, and most preferably from about 100 ms to about 200 ms, at a temperature of 20° C.

It is further preferred that in the second aspect of the invention the filament diameter of the nutritional product decreases less than linearly, and preferably exponentially in time during a CaBER experiment.

In a further preferred embodiment of the second aspect of the invention, the aqueous solution comprises the at least one food grade biopolymer in a concentration of from at least 0.01 wt % to 25 wt %, preferably from at least 0.1 wt % to 15 wt %, and most preferably from at least 1 wt % to 10 wt %.

A further preferred embodiment relates to the method of the second aspect of the invention, further comprising the step of diluting the nutritional product, preferably in an aqueous dilution ranging from 2:1 to 50:1, more preferably from 3:1 to 20:1 and most preferably from 5:1 to 10:1.

In a yet further preferred embodiment of the second aspect of the invention, the method comprises adding to the aqueous solution at least one further food grade biopolymer selected from the group consisting of botanical hydrocolloids, microbial hydrocolloids, animal hydrocolloids, algae hydrocolloids and any combination thereof. It is preferred that the algae hydrocolloids are selected from the group consisting of agar, carrageenan, alginate, or any combinations thereof. In another preferred embodiment, the microbial hydrocolloids are selected from the group consisting of xanthan gum, gellan gum, curdlan gum, or any combinations thereof. In a further preferred embodiment, of the second aspect the botanical hydrocolloids are selected from plant-extracted gums, plant-derived mucilages, or combinations thereof.

In a particularly preferred embodiment of the second aspect of the invention, the method comprises adding to the aqueous solution at least one further food grade biopolymer selected from plant-extracted gums, plant-derived mucilages, or combinations thereof. Preferably, the plant-extracted gums are selected from the group consisting of okra gum, konjac mannan, tara gum, locust bean gum, guar gum, fenugreek gum, tamarind gum, cassia gum, acacia gum, gum ghatti, pectins, modified celluloses (e.g., carboxymethyl cellulose, methyl cellulose, hydroxylpropyl methyl cellulose, hydroxypropyl cellulose), tragacanth gum, karaya gum, or any combinations thereof. It is mostly preferred that the plant-extracted gum is okra gum. In another preferred embodiment, the plant-derived mucilages are selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, or combinations thereof. It is mostly preferred that the plant-derived mucilage is kiwi fruit mucilage and/or cactus mucilage.

In another particularly preferred embodiment of the second aspect of the invention, the method comprises adding to the aqueous solution at least one further food grade biopolymer selected from okra gum and/or kiwi fruit mucilage, or a combination thereof.

A yet further preferred embodiment the invention relates to the method of the second aspect of the invention, further comprising the step of bringing the nutritional product in an administrable form selected from the group consisting of a nutritional formulation, a pharmaceutical formulation, a nutritional supplement, a dietary supplement, a functional food, a beverage product, a full meal, a nutritionally complete formula, and combinations thereof.

The above aspects and their embodiments advantageously provide improved nutritional products, and in particular improved liquid nutritional products.

A particular advantage of these aspects is that improved nutritional products are provided for the treatment of patients suffering from dysphagia.

Yet another particular advantage of the present aspects of the invention is that improved nutritional products are provided that are capable of increasing swallowing-safety of food boluses.

Other aspects, embodiments and advantages of the present invention are described below.

### DETAILED DESCRIPTION OF THE INVENTION

The present invention provides a nutritional product, comprising an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from molecules providing visco-elasticity.

### Nutritional Product

As used herein, the term “nutritional product” includes a nutritional formulation, a pharmaceutical formulation, a nutritional supplement, a dietary supplement, a functional food, a beverage product, a full meal, a nutritionally complete formula, and combinations thereof. Said nutritional product may be in solid, semi-solid or liquid form and may comprise one or more nutrients, foods or nutritional supplements. Preferably, the nutritional product is a liquid product such as a beverage product.

The present inventors have found that providing to dysphagic patients a nutritional product having an increased cohesiveness due to its extensional viscosity, as opposed to the effects of shear viscosity, dramatically reduces the amount of swallowing effort for these patients, as well as the risk of residue build-up in the oropharyngeal and/or esophageal tracts. As such, nutritional products having increased cohesiveness provide improved nutritional intake of dysphagic patients by enabling them to swallow a wider variety of food and beverage products safely and comfortably. This is achieved by improving bolus integrity and thus lending confidence to the patient in being able to consume the different products. The nutritional improvement achieved by an improved food and liquid intake may lead to an overall healthier condition of the patient and prevent further decline.

Therefore, the nutritional product of the present invention is not only modified with regard to its shear viscosity, but with regard to at least one further rheological property such as its cohesiveness.

Shear viscosity is a commonly measured rheological property, which is often referred to as simply viscosity, and which may be determined by any method known in the art. In the present invention, shear viscosity was determined using concentric cylinders in a standard research-grade rheometer (Anton Paar MCR). Said parameter describes the reaction of a material to applied shear stress. In other words, shear viscosity is the ratio between “stress” (force per unit area) exerted on the surface of a fluid, in the lateral or horizontal direction, to the change in velocity of the fluid as you move down in the fluid (a “velocity gradient”).

It is particularly preferred that the nutritional product of the invention has a shear viscosity of at least about 1 mPas, preferably from 5 to 45 mPas, more preferably from 10 to 40 mPas, and most preferably from 20 to 30 mPas, when measured at a shear rate of 50 s-1.

Cohesiveness is a parameter that relates to the ability of a portion of liquid to hold together when being stretched (extended, elongated) in a flow, e.g. passing through a constriction, dewetting of a drop on a surface or thinning of a liquid filament.

In the context of the present disclosure, the relaxation time of a bolus as a measure of its cohesiveness was determined by a Capillary Breakup Extensional Rheometry (CaBER) experiment. The Capillary Breakup Extensional Rheometer is an example for a rheometer applying extensional stress. During the CaBER experiment as performed herein for measuring the relaxation time of the bolus, a drop of said bolus is placed between two vertically aligned and parallel circular metal surfaces, both having a diameter of 6 mm. The metal surfaces are then rapidly separated linearly over a time interval of 50 ms (milliseconds). The filament formed by this stretching action subsequently thins under the action of interfacial tension and the thinning process is followed quantitatively using a laser sheet measuring the filament diameter at its mid-point. The relaxation time in a CaBER experiment is determined by plotting the normalized natural logarithm of the filament diameter during the thinning process versus time and determining the slope of the linear portion (din (D/D0)/dt) of this curve, where D is the filament diameter, DO the filament diameter at time zero and t the time of filament thinning. The relaxation time in this context is then defined as minus one third (−⅓) times the inverse of this slope, i.e. −1/(3dIn(D/D0)/dt).

It is particularly preferred that the nutritional product of the invention has a relaxation time of less than about 2000 ms, preferably from about 20 ms to about 1000 ms, more preferably from about 50 ms to about 500 ms, and most preferably from about 100 ms to about 200 ms, at a temperature of 20° C.

Moreover, preferably, the filament diameter of the nutritional product decreases less than linearly, and more preferably exponentially in time during a CaBER experiment.

In one particularly preferred embodiment, the nutritional product of the invention is a cohesive thin liquid.

A further embodiment relates to the nutritional product in diluted form, preferably in an aqueous dilution ranging from 2:1 to 50:1, more preferably from 3:1 to 20:1 and most preferably from 5:1 to 10:1. By way of example, a dilution of 2:1 means that 1 part of nutritional product is diluted in 2 parts of water.

A further embodiment relates to the nutritional product in administrable form, which may preferably selected from the group consisting of a nutritional formulation, a pharmaceutical formulation, a nutritional supplement, a dietary supplement, a functional food, a beverage product, a full meal, a nutritionally complete formula, and combinations thereof.

“Nutritional compositions,” “pharmaceutical formulations”, “nutritional supplement”, “dietary supplement”, “functional food”, “beverage products”, “full meals”, and/or “nutritionally complete formulas” as used herein, are understood to include any number of optional additional ingredients, including conventional food additives, for example one or more of the following: acidulants, additional thickeners, buffers or agents for pH adjustment, chelating agents, colorants, emulsifiers, excipients, flavor agents, minerals, osmotic agents, pharmaceutically acceptable carriers, preservatives, stabilizers, sugar, sweeteners, texturizers, vitamins, etc. The optional ingredients can be added in any suitable amount.

### Biopolymers

The nutritional product of the present invention comprises an aqueous solution of at least one food grade biopolymer, wherein the at least one food-grade biopolymer is selected from molecules providing visco-elasticity.

It is preferred that the number of food-grade biopolymers in the aqueous solution may be selected from 1 to 10, from 2 to 9, from 3 to 8, from 4 to 7, or from 5 to 6.

Moreover, it is preferred that these biopolymers are comprised in the aqueous solution in a concentration of from at least 0.01 wt % to 25 wt %, preferably from at least 0.1 wt % to 15 wt %, and most preferably from at least 1 wt % to 10 wt %.

As used herein, “wt %” is understood to refer to the weight of polymer per total weight of the product.

As used herein, molecules providing visco-elasticity are understood to include molecules that are long and have a degree of reversible long range structure, such as random coiled polymers, preferably flexible polymers with molecular weight of at least 10'000 g/mol.

In a particularly preferred embodiment, the visco-elasticity providing molecules may be selected from the group consisting of hyaluronic acid, glucosamine sulphate, chondroitin sulphate, collagen, collagen peptides and combinations thereof.

Further, as used herein, collagen peptides are preferably understood to include collagen hydrolysates. Collagen peptides can have a chain length from 2 to maximum 50 amino acids. Preferably, collagen peptides such as Fortigel®, Verisol®, Vitarcal®, etc., are supplied by Gelita AG, Eberbach, Germany.

In one embodiment of the invention, the nutritional product may comprise at least one food-grade biopolymer selected from the above-described molecules providing visco-elasticity plus, in addition, at least one further food grade biopolymer selected from the group consisting of botanical hydrocolloids, microbial hydrocolloids, animal hydrocolloids, algae hydrocolloids and any combination thereof. Thus, in this embodiment, the nutritional product may comprise at least two food-grade biopolymers.

In this embodiment, it is preferred that the total number of food-grade biopolymers in the aqueous solution may be selected from 1 to 10, from 2 to 9, from 3 to 8, from 4 to 7, or from 5 to 6.

Moreover, it is preferred that the total number of food-grade biopolymers together are comprised in the aqueous solution in a concentration of from at least 0.01 wt % to 25 wt %, preferably from at least 0.1 wt % to 15 wt %, and most preferably from at least 1 wt % to 10 wt %.

As used herein, botanical hydrocolloids may preferably be selected from plant-extracted gums, plant-derived mucilages, and combinations thereof.

In the context of this disclosure, plant-extracted gums preferably include any one of okra gum, glucomannans (konjac mannan), galactomannans (tara gum, locust bean gum, guar gum, fenugreek gum), tamarind gum, cassia gum, gum Arabic (acacia gum), gum ghatti, pectins, modified celluloses (e.g., carboxymethyl cellulose, methyl cellulose, hydroxylpropyl methyl cellulose, hydroxypropyl cellulose), tragacanth gum, karaya gum, and combinations thereof. Okra gum is particularly preferred.

Further in this context, plant-derived mucilages are preferably selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, and combinations thereof. In a preferred embodiment, the plant-derived mucilage is kiwi fruit mucilage and/or cactus mucilage.

Preferably, kiwi fruit mucilage is derived from the stem pith of kiwi fruit, which contains about 20% of mucilage and typically represents the plant waste material remaining from kiwi fruit agriculture.

Further in this context, the gums and mucilages are preferably food grade and can be commercially obtained from numerous suppliers.

Alternatively, the above gums and mucilages may be obtained by any suitable extraction method known in the art. For example, gums and mucilages may be extracted by a method comprising the steps of soaking the raw plant material with 10 times of its weight of distilled water and keeping it overnight. A viscous solution is obtained, which is passed through a muslin cloth. The gum or mucilage is precipitated by addition of 95% by weight of ethanol in a ratio of about 1:1 by continuous stirring. A coagulated mass is obtained, which is subsequently dried in an oven at 40 to 45° C., powdered by passing through a sieve and stored in an airtight container.

Further, as used herein, suitable microbial hydrocolloids preferably include xanthan gum, gellan gum, curdlan gum, or combinations thereof.

As used herein, suitable algae hydrocolloids preferably include agar, carrageenan, alginate or combinations thereof. The microbial hydrocolloids may be selected from xanthan gum, gellan gum, curdlan gum, or combinations thereof.

The nutritional product of the invention may also comprise at least one further animal hydrocolloid, which may preferably be selected from hyaluronic acid, glucosamine sulphate, chondroitin sulphate, collagen, collagen peptides, or combinations thereof.

It is particularly preferred that the at least one further food grade biopolymer is selected from botanical hydrocolloids. Most preferably, the at least one further food grade biopolymer is selected from okra gum, cactus mucilage and kiwi fruit mucilage, or any combination thereof.

### Rigid Particles

In a further embodiment of the invention, the aqueous solution of at least one food grade biopolymer may further comprise rigid particles.

In the context of this disclosure, the term “rigid” means that the particles show no measurable deformation under the forces encountered during swallowing.

Preferably, the rigid particles may have a size of from 100 nm to 1 mm, preferably from 200 nm to 900 nm, from 300 nm to 800 nm, from 400 nm to 700 nm, or from 500 nm to 600 nm.

In the context of this disclosure, the particle size is expressed in terms of the average equivalent particle diameter. In the context of this disclosure, the equivalent particle diameter refers to the diameter of a sphere of equal volume as the particle volume, which may be determined by any suitable method known in the art. Preferably, the equivalent particle diameter is determined by laser diffraction, e.g. using a Malvern® Mastersizer instrument. Further, in this context, the average equivalent particle diameter is based on a number average, which is to be understood as the arithmetic mean of all particle diameters in a sample, usually reported as D[1,0].

It is also preferred that the rigid particles are comprised in the aqueous solution in an amount of from 1 to 50% by volume, preferably in an amount of from 5 to 40% by volume, 10 to 30% by volume, or 15 to 20% by volume.

In the context of this disclosure, % by volume signifies the percentage of the volume of all rigid particles in the aqueous solution as a whole, per total volume of said aqueous solution.

In a preferred embodiment, the rigid particles have an elongated shape, which means that they have an aspect ratio of larger than 1.0.

The rigid particles may be comprised of any food grade material, and are preferably selected from sucrose crystals, cocoa particles, coffee particles, mustard particles, microcrystalline cellulose particles, starch and modified starch granules, protein particles, and any combination thereof.

The presence of such rigid particles in the nutritional product of the invention was found to locally enhance extensional flow and to thereby increase extensional stresses, leading to a higher apparent extensional viscosity of said product.

### Further Potential Ingredients

As described above, the nutritional product of the invention may further comprise one or more nutrients, foods or nutritional supplements, which may be selected from the following compounds.

In an embodiment, the nutritional product may further comprise a high molecular weight protein, which is preferably selected from collagen-derived proteins such as gelatin, plant proteins such as potato, pea, lupin, etc., or other proteins of sufficiently high molecular weight (MW=100 kDa and above).

The nutritional product may further comprise a source of dietary protein including, but not limited to animal protein (such as meat protein or egg protein), dairy protein (such as casein, caseinates (e.g., all forms including sodium, calcium, potassium caseinates, casein hydrolysates, whey (e.g., all forms including concentrate, isolate, demineralized), whey hydrolysates, milk protein concentrate, and milk protein isolate), vegetable protein (such as soy protein, wheat protein, rice protein, and pea protein), or combinations thereof. In a preferred embodiment, the protein source is selected from the group consisting of whey, chicken, corn, caseinate, wheat, flax, soy, carob, pea, or combinations thereof.

The nutritional product may further comprise a source of carbohydrates. Any suitable carbohydrate may be used in the bolus of the invention including, but not limited to, sucrose, lactose, glucose, fructose, corn syrup solids, maltodextrin, modified starch, amylose starch, tapioca starch, corn starch or combinations thereof.

The nutritional product may further comprise a source of fat. The source of fat may include any suitable fat or fat mixture. For example, the fat source may include, but is not limited to, vegetable fat (such as olive oil, corn oil, sunflower oil, rapeseed oil, hazelnut oil, soy oil, palm oil, coconut oil, canola oil, lecithins, and the like), animal fats (such as milk fat) or combinations thereof.

The nutritional product may further comprise one or more prebiotics. As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Non-limiting examples of prebiotics include acacia gum, alpha glucan, arabinogalactans, beta glucan, dextrans, fructooligosaccharides, fucosyl lactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional product may further comprise one or more probiotics. As used herein, probiotic micro-organisms (hereinafter “probiotics”) are food-grade micro-organisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. As used herein, the term “micro-organism” is meant to include the bacterium, yeast and/or fungi, a cell growth medium with the micro-organism, or a cell growth medium in which micro-organism was cultivated. The term “food grade micro-organisms” means micro-organisms that are used and generally regarded as safe for use in food. As used herein, a “non-replicating” micro-organism means that no viable cells and/or colony forming units can be detected by classical plating methods. Such classical plating methods are summarized in the microbiology book: James Monroe Jay, et al., Modern food microbiology, 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non-replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

In general, it is believed that probiotic micro-organisms inhibit or influence the growth and/or metabolism of pathogenic bacteria in the intestinal tract. Probiotics may also activate the immune function of the host. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The nutritional product may further comprise one or more amino acids. Non-limiting examples of suitable amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

The nutritional product may further comprise one or more vitamins. As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in minute amounts for normal growth and activity of the body and obtained naturally from plant and animal foods or synthetically made, pro-vitamins, derivatives, analogs.

The nutritional product may further comprise one or more synbiotics, sources of ω-3 fatty acids, and/or phytonutrients and phytochemicals. As used herein, a synbiotic is a supplement that contains both a prebiotic and a probiotic as defined above that work together to improve the microflora of the intestine. Non-limiting examples of sources of w-3 fatty acids such a-linolenic acid (“ALA”), docosahexaenoic acid (“DHA”) and eicosapentaenoic acid (“EPA”), etc., include fish oil, krill, poultry, eggs, or other plant or nut sources such as flax seed, walnuts, almonds, algae, modified plants, etc.

As used herein, “phytonutrients” and “phytochemicals” are non-nutritive compounds that are found in many foods. Phytochemicals are functional foods that have health benefits beyond basic nutrition, and are health promoting compounds that come from plant sources. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, pennyl alcohol); saponins; lipids including: phytosterols (such as, for example, campesterol, beta sitosterol, gamma sitosterol, stigmasterol), tocopherols (vitamin E), and [symbol] -3, -6, and -9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors; vi) other organic acids which include oxalic acid, phytic acid (inositol hexaphosphate); tartaric acid; and anacardic acid; or vii) combinations thereof.

Non-limiting examples of phytonutrients include quercetin, curcumin and limonin and combinations thereof.

The nutritional product may further comprise one or more antioxidants. As used herein, the term “antioxidant” is understood to include any one or more of various substances such as beta-carotene (a vitamin A precursor), vitamin C, vitamin E, and selenium that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include carotenoids, coenzyme Q10 (“CoQ10”), flavonoids, glutathione Goji (wolfberry), hesperidin, lactowolfberry, lignan, lutein, lycopene, polyphenols, selenium, vitamin A, vitamin B1, vitamin B6, vitamin B12, vitamin C, vitamin D, vitamin E, zeaxanthin, or combinations thereof.

The nutritional product may further comprise fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

The nutritional product may further comprise other functional ingredients including chitosans and protein aggregates. Chitosans are linear polysaccharides composed of randomly distributed β-(1-4)-linked D-glucosamine (deacetylated unit) and N-acetyl-D-glucosame (acetylated unit). Among other potential benefits, chitosans have natural antibacterial properties, aid in drug delivery, and are known to rapidly clot blood. Protein aggregates are coalescences of miss-folded proteins driven by interactions between solvent-exposed hydrophobic surfaces that are normally buried within a protein's interior.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof.

For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

### Use

The nutritional product of the invention may preferably be used in treating a swallowing disorder in a patient in need of same.

In the context of the present invention, the term “swallowing disorder” refers to any kind of physiologic dysfunction and/or disorder that is associated with difficulties and/or an impairment of swallowing, and to the symptoms thereof, which in medical terms is referred to as dysphagia, including esophageal and oral pharyngeal dysphagia, and aspiration.

As used herein, the terms “treating”, “treatment” and “to treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treating” and “to treat” are also intended to include the enhancement of one or more primary prophylactic or therapeutic measures. The terms “treatment,” “treating” and “to treat” further intended to include the dietary management of a disease or condition or the dietary management for prophylaxis or prevention a disease or condition.

As used herein, the term “patient” is understood to include a mammal such as an animal and, more preferably, a human that is receiving or intended to receive treatment, as it is herein defined. While the terms “individual” and “patient” are often used herein to refer to a human, the invention is not so limited. Accordingly, the terms “individual” and “patient” refer to any animal, mammal or human having or at risk for a medical condition that can benefit from the treatment.

In this context, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

In a further embodiment, the nutritional products of the invention may be used in promoting safe swallowing of nutritional products, and/or for use in mitigating the risks of aspiration during swallowing of nutritional products. These methods include administering to a patient in need of same the nutritional product of the invention.

### Methods

The present invention further provides a method for making a nutritional product, the method comprising providing an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from a group of molecules providing visco-elasticity, and, optionally, wherein the group of molecules providing visco-elasticity comprises hyaluronic acid, glucosamine sulphate, chondroitin sulphate, collagen, collagen peptides and combinations thereof.

In another aspect, the invention provides a method for improving the cohesiveness of a nutritional product. This method preferably includes adding to a nutritional product an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from a group of visco-elasticity providing molecules, and, optionally, wherein the group of visco-elasticity providing molecules comprises hyaluronic acid, glucosamine sulphate, chondroitin sulphate, collagen, collagen peptides and combinations thereof.

In yet another aspect, the present invention further provides a method for promoting safe swallowing of food boluses. This method preferably includes adding to a nutritional product an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from a group of molecules providing visco-elasticity, and, optionally, wherein the group of molecules providing visco-elasticity comprises hyaluronic acid, glucosamine sulphate, chondroitin sulphate, collagen, collagen peptides and combinations thereof.

In yet another aspect of the invention, a method for treating a patient having a swallowing disorder is provided. This method includes administering to a patient in need of same a nutritional product comprising an aqueous solution of at least one food grade biopolymer capable of providing to the nutritional product: a shear viscosity of less than about 100 mPas, preferably of less than about 50 mPas, when measured at a shear rate of 50 s-1, and a relaxation time, determined by a Capillary Breakup Extensional Rheometry (CaBER) experiment, of more than 10 ms (milliseconds) at a temperature of 20° C., wherein the at least one food-grade biopolymer is selected from a group of molecules providing visco-elasticity, and, optionally, wherein the group of molecules providing visco-elasticity comprises hyaluronic acid, glucosamine sulphate, chondroitin sulphate, collagen, collagen peptides and combinations thereof.

In a preferred embodiment, any one of the above methods may comprise an optional further step of diluting the nutritional product, preferably in an aqueous dilution ranging from 2:1 to 50:1, more preferably from 3:1 to 20:1 and most preferably from 5:1 to 10:1.

In a further preferred embodiment, any one of the above methods may comprise a further step of bringing the nutritional product in an administrable form selected from the group consisting of a nutritional formulation, a pharmaceutical formulation, a nutritional supplement, a dietary supplement, a functional food, a beverage product, a full meal, a nutritionally complete formula, and combinations thereof.

In the above methods, each one of the terms “swallowing disorder”, “nutritional product”, “cohesiveness”, “food grade biopolymer”, “shear viscosity”, “relaxation time”, “molecules providing visco-elasticity”, and “collagen peptides” is preferably defined as set out above.

Most preferably, in the above methods the term “nutritional product” is understood as referring to the nutritional product according to the present invention.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

1. A nutritional product, comprising an aqueous solution of at least one food grade biopolymer capable of providing to the nutritional product:
a shear viscosity of less than about 100 mPas, preferably of less than about 50 mPas, when measured at a shear rate of 50 s⁻¹, and
a relaxation time, determined by a Capillary Breakup Extensional Rheometry (CaBER) experiment, of more than 10 ms (milliseconds) at a temperature of 20° C.,
wherein the at least one food-grade biopolymer is selected from molecules providing visco-elasticity.

2. The nutritional product according to claim 1, wherein the molecules providing visco-elasticity are selected from the group consisting of hyaluronic acid, glucosamine sulphate, chondroitin sulphate, collagen, collagen peptides and combinations thereof.

3. The nutritional product according to claim 1, wherein the shear viscosity is at least about 1 mPas when measured at a shear rate of 50 s⁻¹.

4. The nutritional product according to claim 1, wherein the relaxation time is less than about 2000 ms, preferably from about 20 ms to about 1000 ms at a temperature of 20° C.

5. The nutritional product according to claim 1, wherein the filament diameter of the nutritional product decreases less than linearly during a CaBER experiment.

6. The nutritional product according to claim 1, wherein the aqueous solution comprises the at least one food grade biopolymer in a concentration of from at least 0.01 wt % to 25 wt %.

7. The nutritional product according to claim 1 in diluted form.

8. The nutritional product according to claim 1, comprising at least one further food grade biopolymer selected from the group consisting of botanical hydrocolloids, microbial hydrocolloids, animal hydrocolloids, algae hydrocolloids and any combination thereof.

9. The nutritional product according to claim 8, wherein
the algae hydrocolloids are selected from the group consisting of agar, carrageenan, alginate, and any combination thereof;
the microbial hydrocolloids are selected from the group consisting of xanthan gum, gellan gum, curdlan gum, and any combination thereof:
the botanical hydrocolloids are selected from plant-extracted gums, plant-derived mucilages, and combinations thereof.

10. The nutritional product according to claim 9, wherein the plant-extracted gums are selected from the group consisting of okra gum, konjac mannan, tara gum, locust bean gum, guar gum, fenugreek gum, tamarind gum, cassia gum, acacia gum, gum ghatti, pectins, modified celluloses (e.g., carboxymethyl cellulose, methyl cellulose, hydroxylpropyl methyl cellulose, hydroxypropyl cellulose), tragacanth gum, karaya gum, and any combination thereof.

11. The nutritional product according to claim 9, wherein the plant-derived mucilages are selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, or combinations thereof, and preferably the plant-derived mucilage is kiwi fruit mucilage and cactus mucilage.

12. The nutritional product according to claim 8, wherein the at least one further food grade biopolymer is selected from the group consisting of okra gum and kiwi fruit mucilage, and a combination thereof.

13. The nutritional product according to claim 1 in an administrable form selected from the group consisting of a nutritional formulation, a pharmaceutical formulation, a nutritional supplement, a dietary supplement, a functional food, a beverage product, a full meal, a nutritionally complete formula, and combinations thereof.

14. A method for treating a swallowing disorder, for use in promoting safe swallowing of nutritional products, and/or for use in mitigating the risks of aspiration during swallowing of nutritional products in a patient in need of same comprising administering a composition comprising an aqueous solution of at least one food grade biopolymer capable of providing to the nutritional product: a shear viscosity of less than about 100 mPas, preferably of less than about 50 mPas, when measured at a shear rate of 50 s⁻¹, and a relaxation time, determined by a Capillary Breakup Extensional Rheometry (CaBER) experiment, of more than 10 ms (milliseconds) at a temperature of 20° C., wherein the at least one food-grade biopolymer is selected from molecules providing visco-elasticity.

15. A method for making a nutritional product, the method comprising providing an aqueous solution of at least one food grade biopolymer capable of providing to the nutritional product:
a shear viscosity of less than about 100 mPas, preferably of less than about 50 mPas, when measured at a shear rate of 50 s⁻¹, and
a relaxation time, determined by a Capillary Breakup Extensional Rheometry (CaBER) experiment, of more than 10 ms (milliseconds) at a temperature of 20° C.,
wherein the at least one food-grade biopolymer is selected from a group of molecules providing visco-elasticity, and, optionally, wherein the group of molecules providing visco-elasticity comprises hyaluronic acid, glucosamine sulphate, chondroitin sulphate, collagen, collagen peptides and combinations thereof.
